(19) United States
(12) Patent Application Publication
(10) Pub. No.: US 2008/0053457 A1
(43) Pub. Date: Mar. 6, 2008

(54) Oxygen Delivery Systems

(76) Inventor: Lee McDonald, Barrie (CA)

Correspondence Address: NIKOLAI & MERSEREAU, P.A., 900 SECOND AVENUE SOUTH, SUITE 820, MINNEAPOLIS, MN, 55402, US

(21) Appl. No.: 11/572,267

(22) PCT Filed: Jul. 25, 2005
(86) PCT No.: PCT/CA05/01161

§ 371 Date: Jan. 18, 2007

(30) Foreign Application Priority Data
Jul. 30, 2004 (CA) 2476104

(51) Int. Cl. A61M 16/06

(52) U.S. Cl. 128/207.17; 128/207.18

(57) ABSTRACT

An oxygen delivery system for a patient, the system comprising: (a) an elongated support having ends and constructed to be releasably securable with respect to the patient's head, this support, when in position, extending from side to side with respect to the patient's head; (b) a support arm; (c) means to secure the support arm to one end of the support, for universal relative movement of the arm with respect to the support, and securely positioning the support arm with respect to the support at a desired location; (d) an oxygen delivery tube; (e) patient oxygen delivery means attached to a first end of said oxygen delivery tube; (f) means releasably to attach the oxygen delivery tube and patient oxygen delivery means to the support arm; and (g) means releasably to attach a second end of the oxygen delivery tube to an oxygen source; when the elongated support is in position, the support arm to support the oxygen delivery tube and patient oxygen delivery means in proper position for supplying oxygen to the patient.

### FIELD OF THE INVENTION

The present invention relates to a novel system for delivery of oxygen to a patient and more particularly to a system which can be used to replace conventional oxygen masks and nose cannula oxygen delivery systems.

### BACKGROUND OF THE INVENTION

Conventional oxygen masks comprise tent like structures which are strapped over the nose and mouth of the patient, often using an elastic band or bands behind the patient's ears or head. Oxygen is fed from a supply through a tube into the bottom portion of the mask at the front of the patient. Many problems exist with such masks, including the fact that many patients find them claustrophobic, the mask must be removed for the patient to speak or eat, thereby discontinuing therapy and the face mask creates irregular infusion of oxygen by the patient since exhaled air from the patient is mixed with oxygen in the mask.

Conventional nose cannula oxygen delivery systems employ an oxygen delivery tube with tubular, open ended nasal prongs at the delivery end of the tube for insertion into a patient's nasal passages. The oxygen delivery tube and nasal cannula are supported in position by a tube wrapped about the patient's ears or head, making the system both difficult to handle and uncomfortable since it applies downward pressure on the patient's ears when the patient is in a seated position. As well, patients often get nose bleeds from the dryness of the nasal cannula.

Of background interest is Ketchedjian, U.S. Pat. No. 6,247,470 issued Jun. 19, 2001 which describes and illustrates an oxygen delivery apparatus comprising a headset to which is pivotally attached, for rotation in one plane, a flexible arm carrying tubular members for passing oxygen to a patient's mouth. The apparatus is also provided with a carbon dioxide monitoring system.

McCombs et al., U.S. Pat. No. 6,065,473 issued May 23, 2000 describes a somewhat similar apparatus, for non-medical purposes, intended to dispense concentrated oxygen to users, the apparatus comprising an oxygen delivery nozzle attached by an arm extending from a flexible head band, to bathe the user's nose and mouth with oxygen, when in use. Laid-open German Application DE 43 07 754 A1, published Apr. 7, 1994, teaches a system for controlled supply or removal of respiratory air from a user, which system incorporates a mask body held by a rigid air tube over the mouth and/or nose of the user, the air tube being pivotally adjustable in one plane, to enable proper position of the mask.

U.S. Pat. No. 3,683,907 of Cotabish issued Aug. 15, 1972 describes and illustrates a fresh air respirator, for use for example by miners, which comprises a cup, supported by pivotable arms in front of the face of the user, a stream of air being conducted to the cup to provide fresh air around the user's nose and mouth.

The applicant has developed a number of lightweight oxygen delivery systems for patients, as described for example in U.S. Pat. Nos. 6,675,796 issued Jan. 13, 2004, 6,595,207 issued Jul. 22, 2003 and 6,450,166 issued Sep. 17, 2002. Also, applicant's U.S. Design Pat. Nos. D449,376 issued Aug. 16, 2003 and D449,883 issued Oct. 30, 2001 illustrate designs for such devices. All of these references feature oxygen diffuser devices, designed to create a turbulent oxygen flow, to be situated during use in front of the nose and mouth of a patient, and being held in that area by means of a mount such as a head band, to which is secured a rigid, but bendable oxygen delivery tube. In one embodiment of the apparatus described and illustrated in U.S. Pat. No. 6,450,166, the support tube may swivel in one plane to assist in proper positioning of the diffuser.

Other references of general background interest include U.S. Pat. No. 4,282,869 of Zidulka issued Aug. 11, 1981, U.S. Pat. No. 4,018,221 of Rennie issued Apr. 19, 1977, U.S. Pat. No. 5,687,715 of Landis et al. issued Nov. 18, 1997, U.S. Pat. No. 4,465,067 of Koch et al. issued Aug. 4, 1984 and U.S. Pat. No. 5,697,363 of Hart issued Dec. 16, 1977, all of which describe and illustrate different types of head mounted apparatus for delivering oxygen or other gases to a patient.

Most of these prior art devices intended for delivery of oxygen to a patient do not provide the ease of usage, by both health care workers and the patient, and reliability, against unintended removal, as is required to permit wide spread use by the health care professional. As well, cost and economy are important factors not addressed by these prior art devices, most of which provide a single function and will normally be discarded entirely after use by one patient.

It is an object of the present invention to provide a more versatile, more economical and more practical system for delivery of oxygen to patients.

### SUMMARY OF THE INVENTION

In accordance with the present invention there is provided a patient oxygen delivery system. The system comprises an elongated support having ends and constructed to be releasably securable with respect to the patient's head. This support, when in position, extends from side to side with respect to the patient's head. A support arm is provided, together with means to secure the support arm to one end of the support, for universal relative movement of the arm with respect to the support, and securely to position the support arm with respect to the support at a desired location. An oxygen delivery tube is also provided and a patient oxygen delivery means is attached to a first end thereof. Means are provided releasably to attach the oxygen delivery tube and patient oxygen delivery means to the support arm. As well, means are provided releasably to attach a second end of the oxygen delivery tube to an oxygen source. When the elongated support is in position, the support arm supports the oxygen delivery tube and patient oxygen delivery means in a proper position for supplying oxygen to the patient.

In one embodiment of the present invention the oxygen delivery means is nasal cannula secured to the first end of the oxygen delivery tube and communicating therewith so as to deliver oxygen to the patient during operation of the system.

In an alternative embodiment the oxygen delivery means is an oxygen diffuser unit secured to the first end of the oxygen delivery tube, the unit comprising a body having a wall with an interior surface of generally concave configuration, circumscribing a centrally positioned oxygen outlet communicating with the oxygen delivery tube and opening into the concave interior surface in operation so as to direct the flow of oxygen from the oxygen outlet generally towards a patient's nose and mouth. A baffle is situated within the concave wall and located in a path of an oxygen stream exiting the oxygen outlet during operation of the diffuser unit so as to generate turbulence to assist in mixing of oxygen with ambient air and create a mushroom shaped plume of oxygen enriched air.

It is preferred that the means to secure the support arm to the elongated support be a ball and socket arrangement.

The elongated support for releasably securing to the patient's head may be either a curved resilient head band or an elongated resilient tube, the tube being configured so as to be seated during operation of the system behind the patient's head and over the patient's ears.

The oxygen delivery system according to the present invention provides an extremely versatile, easy-to-use, economical and comfortable system for delivery of oxygen to a patient. The system is readily adaptable to different sizes of patients. Its interconnectable construction permits multiple arrangements, using many of the same components, for different patients.

### BRIEF DESCRIPTION OF THE DRAWINGS

These and other advantages of the invention will become apparent upon reading the following detailed description and upon referring to the drawings in which:—

FIG. 1 is a perspective view of one embodiment of the oxygen delivery system featuring an oxygen diffuser according to the present invention in position on the head of a patient;

FIG. 2 is a section view of the diffuser unit of FIG. 1, along lines 2-2 of FIG. 1;

FIG. 3 is a perspective view of an alternative embodiment of the system of the present invention illustrating a nasal cannula arrangement;

FIG. 4 is a perspective view of yet a further embodiment of the present invention featuring an alternative arrangement of oxygen diffuser unit incorporating a carbon dioxide monitor unit;

FIG. 5 is an section view of a slightly modified version of the diffuser unit of FIG. 4 along line 5-5 of FIG. 4; and

FIGS. 6 and 6a are fragmentary perspective views of an arrangement of the ball and joint connection according to the present invention.

While the invention will be described in conjunction with illustrated embodiments, it will be understood that it is not intended to limit the invention to such embodiments. On the contrary, it is intended to cover all alternatives, modifications and equivalents as may be included within the spirit and scope of the invention as defined by the appended claims.

### DETAILED DESCRIPTION OF THE PREFERRED EMBODIMENTS

In the following description, similar features in the drawings have been given similar reference numerals.

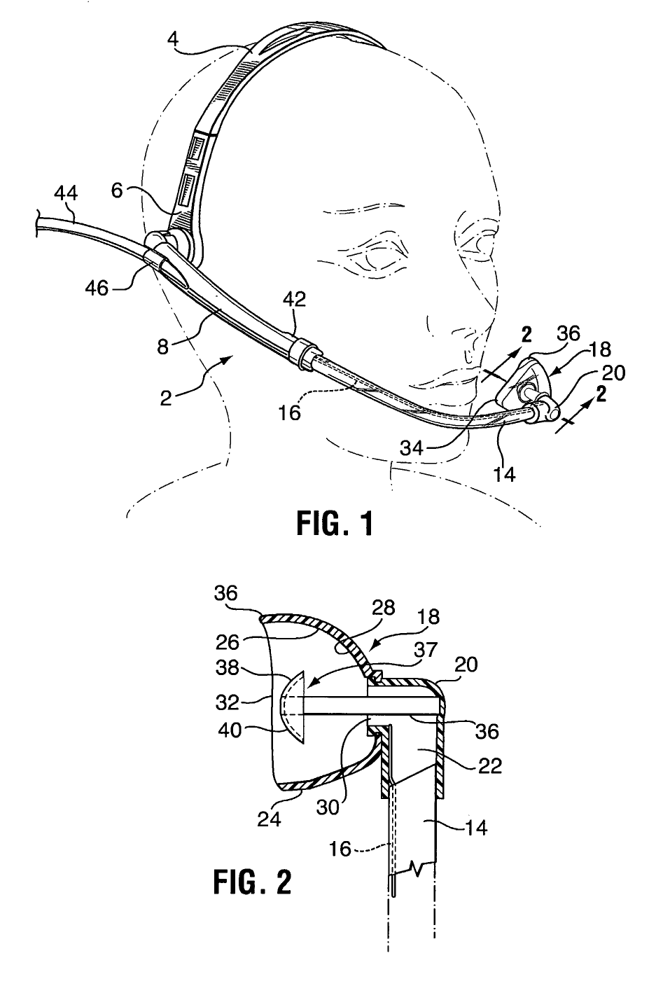

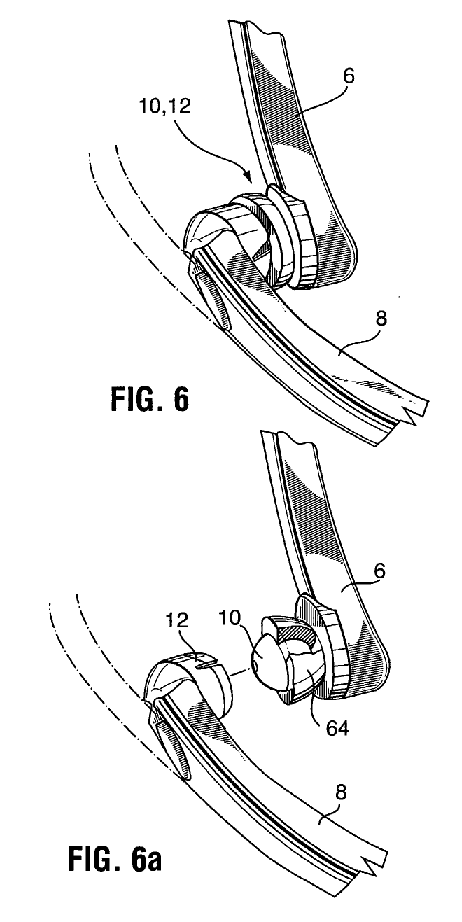

Turning to FIG. 1 there is illustrated a patient oxygen delivery system 2. The system includes an elongated support in the form of head band 4 which is somewhat flexible and constructed so as to be releasably securable with respect to the patient's head. The ends 6 of head band 4 are intended to comfortably sit on either side of the patient's head as illustrated. A hollowed support arm 8 is pivotally attached to one of the ends 6 of head band 4, so as to be universally movable relative to head band 4. A ball 10 and joint 12 attachment (FIG. 6a) accomplishes this end. There is sufficient frictional engagement of ball 10 within joint 12 to enable secure positioning of the support arm with respect to head band 4 in a particular desired location.

A flexible oxygen delivery tube 14 is provided, having a wire 16 embedded in it so that this tube is bendable to a particular shape and will maintain that shape. A conventional diffuser unit 18 serves as the oxygen delivery means for this embodiment of the present invention. Diffuser unit 18 is secured to one end of oxygen delivery tube 14 and comprises a rigid elbow 20, provided with an oxygen delivery passageway 22 extending from one end of the elbow to the other. Oxygen delivery tube 14 communicates with that passageway. Diffuser body 24 is rotatably secured to the other end of elbow 20 as illustrated (FIG. 2), body 24 having a wall 26 of cup shaped appearance, extending from a base 28 which circumscribes an oxygen outlet 30 which in turn communicates with the oxygen delivery passageway 22 of elbow 20. Wall 26 extends from that base, flaring outwardly to an edge 32 of triangular peripheral contour. The peripheral corners 34 are rounded, with one of the corners 36, intended to be the uppermost corner when in use, and the proximal portions of the wall edge 32, being raised with respect to the other corners and their proximal wall edge portions as illustrated, to facilitate the direction of oxygen towards a patient's nose and mouth. This construction, with protruding corner 36 and proximal edges of the wall 26 being positioned proximal to the patient's nose when in use, and the wider triangular side at the bottom proximal to a patient's mouth, provides optimal oxygen delivery to a patient.

A mushroom-shaped baffle 37 is seated over the oxygen outlet 30 so as to assist in generating turbulence and assist in the diffusion of oxygen and avoid a direct flow of oxygen towards a patient's face. This baffle 37 comprises a post 36 centrally seated in body 24 with respect to outlet 30, having at its top a head 38 with a curled back conical skirt 40. This construction of baffle 37 impedes oxygen flow from the rear of diffuser body 24 inducing the transmission of oxygen from jet to turbulent flow.

Oxygen delivery tube 14 is releasably secured within the hollow part of support arm 8 by clips 42. It may be releasably attached to an oxygen supply tube 44, going to an oxygen source (not illustrated) by means of attachment means 46. Alternatively, diffuser 18 may be secured to the other end of oxygen delivery tube 14 by means of releasable attachment means (not illustrated). In either of these ways, the oxygen delivery means may be replaced or substituted with one of a different construction (along lines to be described subsequently).

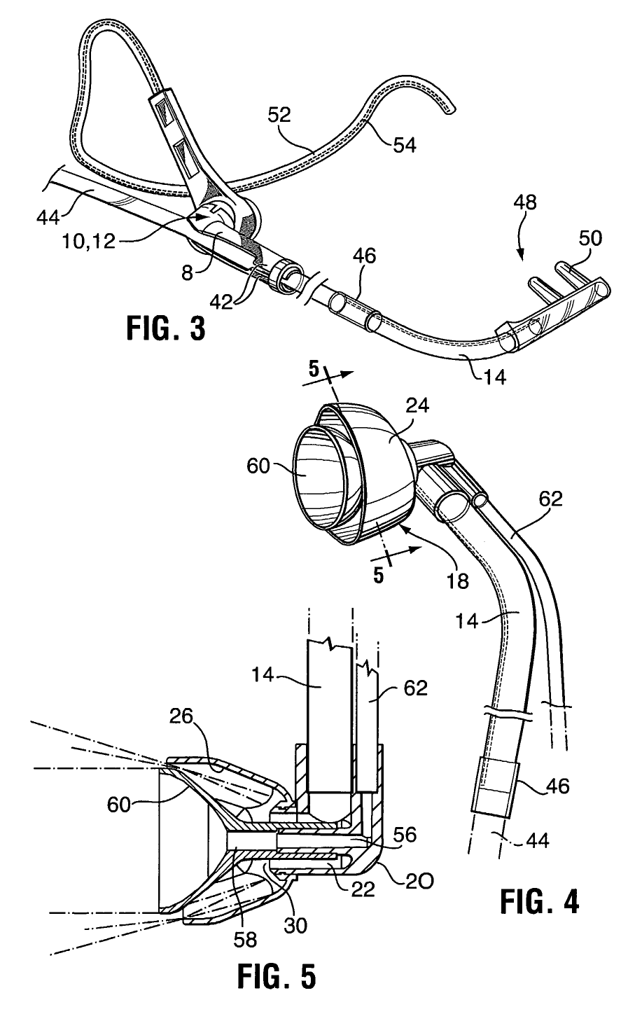

An alternative construction of oxygen delivery means is the nasal cannula 48 of FIG. 3. This is a conventional cannula with hollow nasal prongs 50 for inserting in a patient's nose, through which oxygen flows to the patient. Cannula 48 is secured to one end of an oxygen delivery tube 14 as illustrated, the other end of tube 14 being releasably secured by attachment means 46 to the oxygen supply tube 44 from the oxygen source (not illustrated). Also, as with the diffuser 18 of FIGS. 1 and 2, cannula 48 may itself be releasably attached to the other end of delivery tube 14 so it can be detached from the oxygen delivery tube 14. In these ways the cannula 48, or cannula and oxygen delivery tube 14 can be replaced.

As well oxygen delivery tube 14 and nasal cannula 48 are supported, instead of on a head band 4, on a contoured flexible tube 52 of U-shaped configuration, designed to sit behind the user's head with its ends resting over the user's ears, is provided. This tube may for example be similar to the oxygen delivery tube 14 in that it is made from flexible plastic in which is embedded a positioning wire 54 which enables the tube to be bent into an appropriate shape and to maintain that shape. As well, it is preferred that the tube be coated with a friction enhancing substance to facilitate the maintaining of this tube in position on a patient. Again a universal ball 10 and joint 12 connection secures support arm 8 to one end of tube 52.

In FIGS. 4 and 5, yet an alternative embodiment of diffuser 18 is illustrated. While the diffuser body 24 is similar to that of FIGS. 1 and 2, elbow 20 contains not only a passageway for oxygen, but also a passageway 56 for carbon dioxide monitoring. Elbow 20 is secured (either releasably or not) to one end of oxygen delivery tube 14. The interior surface of the diffuser body 24 again circumscribes the oxygen outlet 30 of the diffuser, and directs the flow of oxygen generally outwardly from the diffuser 18. Releasable attachment means 46 may be provided at the other end of oxygen delivery tube 14, so that this diffuser 18 and oxygen delivery tube 14 can be removed and replaced with another appropriate oxygen delivery tube and oxygen delivery means.

However, also within the cup-shaped wall 26 of diffuser 18 is centrally positioned a carbon dioxide intake 58 formed of a concave, preferably hemispheric-shaped (FIG. 4) or conical (FIG. 5) wall 60. The carbon dioxide intake 58, as can be seen in FIG. 5, fills a significant part of the interior of the diffuser body 24 as is the case with the diffuser 18 of FIGS. 1 and 2, to permit greater flexibility of the diffuser unit 18, body 24 and carbon dioxide intake wall 60 are secured to one end of elbow 20 so as to rotate 360° on it. In this way, the proper orientation of diffuser body 24 with respect to the patient's nose and mouth can be achieved.

Wall 60 of carbon dioxide intake 58 circumscribes carbon dioxide 58 which communicates with carbon dioxide monitor passageway 56 formed in elbow 20. Carbon dioxide monitor passageway 56 is separate from and does not communicate with oxygen delivery passageway 22 in elbow 20. At the other end of carbon dioxide monitor passageway 56 is a carbon dioxide monitor tube 62, that tube to communicate with a carbon dioxide monitor (not shown) when the system is in use. In this manner, carbon dioxide exhaled by the patient can be collected in the vicinity of the patient's nose and mouth by carbon dioxide intake 58, and passed to the carbon dioxide monitor, while at the same time oxygen is being delivered from an oxygen source (again not shown), through oxygen delivery tube 14 and oxygen diffuser 18 to the patient's nose and mouth area. As can be seen in FIGS. 4 and 5, it is preferable that the carbon dioxide intake wall 40 extends outwardly beyond the edges of the diffuser body wall 26. It has been found in tests that this particular construction of diffuser body and carbon dioxide intake wall provides a baffle for generating the necessary oxygen turbulence to provide an effective plume of oxygen for delivery to the nose and mouth area of a patient, while at the same time it enables an effective carbon dioxide monitoring of the patient's exhaled breath.

While the ball 10 and socket 12 arrangement illustrated more particularly in FIGS. 6 and 6a is normally suitable to maintain the support arm 8, oxygen delivery tube 14 and oxygen delivery means in appropriate positioning for use by a patient, a hemispherical deformable washer 64 of friction-creating material may be seated at the base of the ball, with its concave surface seatably engaging confronting portions of the exterior of the socket which fit over the ball. In this way, more rigidity can be achieved in the ball and socket arrangement, once the arm has been properly positioned, so that it is more difficult to move the arm from that relative position with respect to the head support.

It will be appreciated that the oxygen delivery system 2 in accordance with the present invention provides a basic arrangement (head support, support arm and universal joint between these two) which readily enables different types of oxygen delivery means such as cannulas or oxygen diffusers, to be used and replaced or changed as required. Thus, a kit having either a head band 4 for or behind-the-head-tube 56, with universally movably secured support arm 8, can be provided with multiple types of oxygen delivery means such as different types of diffuser units 18 and cannulas 48 releasably attached to an oxygen supply tube 44 and held by support arm 8. Besides its versatility, the system according to the present invention permits tremendous savings in inventories in hospitals and other health care facilities, since components such as the head support and support arm units may be reused or configured with different oxygen delivery means, as necessitated by patient requirements.

Thus, it is apparent that there has been provided in accordance with the invention a patient oxygen delivery system that fully satisfies the objects, aims and advantages set forth above. While the invention has been described in conjunction with illustrated embodiments thereof, it is evident that many alternatives, modifications and variations will be apparent to those skilled in the art in light of the foregoing description. Accordingly, it is intended to embrace all such alternatives, modifications and variations as fall within the spirit and broad scope of the invention.

1. An oxygen delivery system for a patient, the system comprising:
(a) an elongated support having ends and constructed to be releasably securable with respect to the patient's head, this support, when in position, extending from side to side with respect to the patient's head;
(b) a support arm;
(c) means to secure the support arm to one end of the support, for universal relative movement of the arm with respect to the support and securely positioning the support arm with respect to the support at a desired location;
(d) an oxygen delivery tube;
(e) patient oxygen delivery means attached to a first end of said oxygen delivery tube;
(f) means releasably to attach the oxygen delivery tube to the support arm; and
(g) means releasably to attach a second end of the oxygen delivery tube to an oxygen source;
when the elongated support is in position, the support arm to support the oxygen delivery tube and patient oxygen delivery means in proper position for supplying oxygen to the patient.

2. A system according to claim 1, wherein the patient oxygen delivery means comprises a nasal cannula secured to said first end of the oxygen delivery tube and communicating therewith so as to deliver oxygen to the patient during operation of the system.

3. A system according to claim 2, wherein the oxygen delivery tube is made of flexible plastic with a wire embedded therein so as to make the tube bendable to a particular shape and capable of maintaining that shape.

4. A system according to claim 1, wherein the patient oxygen delivery means comprises an oxygen diffuser unit secured to said first end of the oxygen delivery tube, said unit comprising a body having a wall with an interior surface of generally concave configuration, circumscribing a centrally positioned oxygen outlet communicating with said oxygen delivery tube and opening into the concave interior surface, in operation so as to direct the flow of oxygen from the oxygen outlet generally towards a patient's nose and mouth, and a baffle situated within the concave wall and located in a path of an oxygen stream exiting the oxygen outlet during operation of the diffuser unit so as to generate turbulence to assist in mixing of oxygen with ambient air and create a mushroom shaped plume of oxygen enriched air.

5. A system according to claim 4, wherein the oxygen delivery tube is made of flexible plastic with a wire embedded therein so as to make the tube bendable to a particular shape and capable of maintaining that shape.

6. A system according to claim 4, wherein the baffle comprises a post, an end of which is seated centrally with respect to the oxygen outlet, to the other end of which post is secured a head with a curled back conical skirt, the under side of the skirt being located in the path of the oxygen stream exiting the oxygen outlet during operation of the diffuser so as to generate the turbulence.

7. A system according to claim 4, wherein the baffle has a concave shaped wall and is centrally secured within the diffuser body wall, a carbon dioxide intake positioned within the concave shaped wall of the baffle, and the diffuser unit being further provided with a carbon dioxide monitor tube communicating with the carbon dioxide intake for passing oxygen exhaled by the patient, during operation of the system, to a carbon dioxide monitor.

8. A system according to claim 1, wherein the elongated support for releasable securing to the patient's head comprises a curved resilient head band.

9. A system according to claim 1, wherein the elongated support for releasable securing to the patient's head comprises an elongated resilient tube, the tube being configured so as to be seated during operation of the system, behind the patient's head and over the patient's ears.

10. A system according to claim 9, wherein the elongated resilient tube is constructed so as to be bendable to a particular shape and capable of maintaining that shape.

11. A system, according to claim 2, wherein the elongated support for releasable securing to the patient's head comprises a curved resilient head band.

12. A system according to claim 2, wherein elongated support for releasable securing to the patient's head comprises an elongated resilient tube, the tube being configured so as to be seated, during operation of the system, behind the patient's head and over the patient's ears.

13. A system according to claim 4, wherein the elongated support for releasable securing to the patient's head comprises a curved resilient head band.

14. A system according to claim 4, wherein elongated support for releasable securing to the patient's head comprises an elongated tube, the tube being configured so as to be seated, during operation of the system, behind the patient's head and over the patient's ears.

15. A system according to claim 1, wherein the means for securing the support arm to the elongated support is a ball and socket arrangement.

16. A system according to claim 15, wherein a friction generation washer is seated between the ball and socket so as to reduce unintended movement of the ball and socket when the support arm is in a desired location with respect to the support.

17. A kit for oxygen delivery to a patient, the kit comprising:
(a) an elongated support having ends and constructed to be releasably securable with respect to the patients head, this support, when in position, extending from side to side with respect to the patient's head;
(b) a support arm;
(c) means to secure the support arm to one end of the support, for universal relative movement of the arm with respect to the support, and securely positioning the support arm with respect to the support at a desired location;
(d) one or more patient oxygen delivery means selected from the group comprising: a nasal cannula secured to a first end of an oxygen delivery tube and communicating therewith so as to deliver oxygen to the patient during operation of the system, and an oxygen diffuser unit secured to a first end of an oxygen delivery tube, said unit comprising a body having a wall with an interior surface of generally concave configuration, circumscribing a centrally positioned oxygen outlet communicating with said oxygen delivery tube and opening into the concave interior surface, in operation so as to direct the flow of oxygen from the oxygen outlet generally towards a patient's nose and mouth, and a baffle situated within the concave wall and located in a path of an oxygen stream exiting the oxygen outlet during operation of the diffuser unit so as to generate turbulence to assist in mixing of oxygen with ambient air and create a mushroom shaped plume of oxygen enriched air;
(e) means releasably to attach one of the oxygen delivery means to the support arm; and
(f) means releasably to attach a second end of the oxygen delivery tube to an oxygen source;
when the elongated support is in position, the support arm to support the oxygen delivery tube and patient oxygen delivery means in proper position for supplying oxygen to the patient.

18. A kit according to claim 17, wherein the elongated support for releasable securing to the patient's head comprises a curved resilient head band.

19. A kit according to claim 17, wherein the elongated support for releasable securing to the patient's head comprises an elongated resilient tube, the tube, the tube being configured so as to be seated, during operation of the system, behind the patient's head and over the patient's ears.

20. A kit according to claim 19, wherein the elongated resilient tube is constructed so as to be bendable to a particular shape and capable of maintaining that shape.
